(19) United States
(12) Patent Application Publication
(10) Pub. No.: US 2003/0144227 A1
(43) Pub. Date: Jul. 31, 2003

(54) Pharmaceutical composition comprising Nit2 for use in inhibiting tumor cell growth

(75) Inventors: Chin-Hsiang Chien, Taipei (TW); Chun-Hung Lin, Taipei (TW)

Correspondence Address: Ladas & Parry, 26 West 61st Street, New York, NY, 10023, US

(73) Assignee: NATIONAL YANG MING UNIVERSITY

(21) Appl. No.: 10/173,180

(22) Filed: Jun. 17, 2002

(30) Foreign Application Priority Data
Jan. 31, 2002 (TW) 091101840

(51) Int. Cl. A61K 48/00; A61K 38/00

(52) U.S. Cl. 514/44; 514/2

(57) ABSTRACT

The present invention relates to new use of Nit2 gene in the inhibition of tumor cell growth. In particular, the invention provides the pharmaceutical compositions for inhibiting tumor cell growth comprising Nit2 gene and Nit2 protein respectively.

### BACKGROUND OF THE INVENTION

1. Field of the Invention

The present invention relates to new use of Nit2 gene in the inhibition of tumor cell growth.

2. Description of the Prior Art

Apoptosis, also called “programmed cell death,” is a carefully regulated network of biochemical events which act as a cellular suicide program aimed at removing irreversibly damaged cells. Mutations or cellular events which potentiate apoptosis may result in premature cell death. Studies have been conducted to explore the possibility that tumor cells could be eliminated by artificially triggering apoptosis. Overexpression of tumor suppressor genes such as p53 gene and RB gene, may induce the apoptotic process of cancer cells. The main function of p53 protein is to be an activator of the transcription of some genes that is capable of, by a process not yet well defined, blocking the cell in the G1 phase of the cell cycle during the appearance of mutations, during the replication of the genome, and triggering a number of DNA repair processes. Furthermore, in the event of a malfunctioning of these repair processes or in the event of the appearance of mutation events which are too many to be corrected, this protein is capable of inducing the phenomenon of apoptosis. The retinoblastoma (RB) gene is a tumor suppressor gene and an effective candidate target for gene therapy approach. Such tumor suppressor genes have been used as tools of gene therapy.

The Nit gene of bacteria and plants encodes the nitrilase that degrades the nitrites to carboxylic acid and ammonia. In C. elegans and Drosophila, the Nit and Fhit together form a fusion protein called NitFhit, in which the domain in N terminal is Nit and the domain in C terminal is Fhit (Pekarsky et al., 1998, Proc. Natl. Acad. Sci. USA., 95:8744-8749). In mammalians, Nit and Fhit are independently expressed proteins. Fhit protein is not only a hydrolase for diadenosine but also a tumor suppressor gene (Sard et al., 1999, Proc. Natl. Acad. Sci. USA., 96:8489-8492). However, the function of human Nit gene family such as Nit1 and Nit2 has not been identified. Neither have the function of the Nit gene and the use thereof.

### SUMMARY OF THE INVENTION

The invention relates to a method of inhibiting tumor cell growth in a mammal comprising the step of contacting a tumor cell with a Nit2 gene in an amount effective to inhibit tumor cell growth.

The invention also relates to a pharmaceutical composition for use in inhibiting tumor cell growth, which comprises a Nit2 gene and a pharmaceutically acceptable carrier.

The invention also relates to a pharmaceutical composition for use in inhibiting tumor cell growth, which comprises a Nit2 protein and a pharmaceutically acceptable carrier.

### BRIEF DESCRIPTION OF THE DRAWINGS

FIG. 1 shows the construction of pEGFP-C1-NIT2 plasmid.

FIG. 2 shows the fluorescence photos for the expression of Nit2 gene and Fhit gene in the OVCAR-3 cells.

FIG. 3 shows the growth curve of the OVCAR-3 cells transfected with Nit2 and Fhit genes.

FIG. 4 shows FACS analysis of the percentage of G₀/G₁ phase of OVCAR-3 cells transfected by pEGFP-C1-NIT2. The cell cycle was determined by the flow cytometry.

(a) OVCAR-3 was transfected by pEGFP-C1 plasmid.

(b) OVCAR-3 was transfected by pEGFP-C1-NIT2 plasmid.

### DETAILED DESCRIPTION OF THE INVENTION

The present invention relates to a new use of Nit2 gene in inhibiting the growth of cancer cells. The new function of Nit2 gene is identified by the disclosures of the present invention.

The invention is directed to a method of inhibiting tumor cell growth in a mammal comprising the step of contacting a tumor cell with a Nit2 gene in an amount effective to inhibit tumor cell growth.

The invention is directed to a method of inhibiting tumor cell growth in a mammal comprising the step of contacting a tumor cell with a Nit2 protein in an amount effective to inhibit tumor cell growth.

According to the invention, the phrase “tumor cells” as used herein are interchangeable with “cancer cells” and “tumors” and include, but are not limited to, autografts, allografts, syngeneic, non-syngeneic and xenografts. Tumor cells include any type of cell that, upon apoptosis, induces resistance to tumor growth, including but not limited to tumor cells. Any type of tumor or cancer cells that undergoes apoptosis and induces resistance to tumor growth, is useful in the present invention. Tumors which are treatable with the methods of the present invention may be primary or secondary, benign, malignant, metastatic, or micrometastatic tumors. Tumors treatable with the methods of the present invention include, but are not limited to, melanoma, prostate, ovary, mammary, pancreatic, lungs, colon, and smooth muscle tumors, as well as cells from glioblastoma, bone marrow stem cells, hematopoietic cells, osteoblasts, epithelial cells, fibroblasts, as well as any other tumor cells that undergo apoptosis and induce resistance to tumor cells.

According to the invention, the term “mammal” as used herein includes but is not limited to, Order Rodentia, such as mice; Order Logomorpha, such as rabbits; more particularly Order Carnivora, including Felines (cats) and Canines (dogs); particularly Order Artiodactyla, Bovines (cows) and Suines (pigs); and Order Penrssodactyla, including Equines (horses); and most particularly Order Primates, Ceboids and Simoids (monkeys) and Anthropoids (humans and apes). Most preferably, the mammals are humans.

According to the invention, the term “Nit2 gene” as used herein refers to any DNA sequence or the fragment thereof that has the function equivalent to the natural Nit2 gene. For example, the DNA sequence includes: (a) the DNA analog sequence derived from coding regions of the natural Nit2 gene; or (b) the DNA analog sequence capable of hybridization of DNA sequences of (a) under moderately stringent conditions; or (c) the DNA sequences which are degenerative as a result of the genetic code to the DNA analog sequences defined in (a) or (b). More preferably, the Nit2 gene is a human Nit2 gene. Most preferably, the Nit2 gene has the sequence of SEQ ID NO: 1:

| | | |
|---|---|---|
| 1 | gtggtgcttg | tctgcagagt catgacctct ttccgcttgg ccctcatcca gcttcagatt |
| 61 | tcttccatca aatcagataa cgtcaccgc gcttgtagct tcatccggga ggcagcaacg | |
| 121 | caaggacca aaatagtttc tttgccggaa tgctttaatt ctccatatgg agcgaaatat | |
| 181 | tttcctgaat atgcagagaa aattcctggt gaatccacac agaagctttc tgaagtagca | |
| 241 | aaggaatgca gcatatatct cattggaggc tctatccctg aagaggatgc tgggaaatta | |
| 301 | tataacacct gtgctgtgtt tgggcctgat ggaactttac tagcaaagta tagaaagatc | |
| 361 | catctgtttg acattgatgt tcctggaaaa attacatttc aagaatctaa aacattgagt | |
| 421 | ccgggtgata gtttctccac atttgatact ccttactgca gagtgggtct gggcatctgc | |
| 481 | tacgacatgc ggtttgcaga gcttgcacaa atctacgcac agagaggctg ccagctgttg | |
| 541 | gtatatccag gagctttaa tctgacc act ggaccagccc attgggagtt acttcagcga | |
| 601 | agccgggctg ttgataatca ggtgtatgtg gccacagcct ctcctgcccg ggatgacaaa | |
| 661 | gcctcctatg ttgcctgggg acacagcacc gtggtgaacc cttgggggga ggttctagcc | |
| 721 | aaagctggca cagaagaagc aatcgtgtat tcagacatag acctgaagaa gctggctgaa | |
| 781 | atacgccagc aaatccccgt ttttagacag aagcgatcag acctctatgc tgtggagatg | |
| 841 | aaaaagccct aaagtttatg tttctaatgt gtcacagaat aggacgatat gattctacaa | |
| 901 | cataatcaac tccctattaa attctttaat gaagaaaaaa aatttaaaaa aaaaaaaaaa | |
| 961 | aa | |

According to the invention, the Nit2 proteins are those encoded by the Nit2 gene as described therein. Preferable, the Nit2 protein is that encoded by the sequence of SEQ ID NO: 1 or the degenerate sequence thereof.

According to the invention, gene transfer systems known in the art may be useful in the delivery of the Nit2 gene of the present invention. These include viral and nonviral transfer methods. A number of viruses have been used as gene transfer vectors, including papovaviruses, e.g., SV40, adenovirus, vaccinia virus, adeno-associated virus, herpes viruses including HSV and EBV, lentiviruses, Sindbis and Semliki Forest virus, and retroviruses of avian, murine, and human origin. Most human gene therapy protocols have been based on disabled murine retroviruses, although adenovirus and adeno-associated virus have also been used.

Nonviral gene transfer methods known in the art include chemical techniques such as calcium phosphate coprecipitation; mechanical techniques, for example microinjection; membrane fusion-mediated transfer via liposomes or protein vector; and direct DNA uptake and receptor-mediated DNA transfer. Viral-mediated gene transfer can be combined with direct in vivo gene transfer using liposome delivery, allowing one to direct the viral vectors to the tumor cells and not into the surrounding nondividing cells. Alternatively, the retroviral vector producer cell line can be injected into tumors. Injection of producer cells would then provide a continuous source of vector particles. This technique has been approved for use in humans with inoperable brain tumors.

According to the invention, the Nit2 gene or Nit2 protein as used in the methods of the invention is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners.

In some embodiments of the present invention, the methods of the invention can be used in combination with other anti-cancer therapies. These other therapies may be known at the time of this application, or may become apparent after the date of this application. Nit2 gene or Nit2 protein may be used in combination with other therapeutic polynucleotides, polypeptides or chemotherapeutic agents. For example, Nit gene or Nit2 protein may be used in conjunction with other known polypeptides, such as TNF-alpha, P53 or RB. Nit2 gene or Nit2 protein may be used in conjunction with any suitable chemotherapeutic agent. In one representative embodiment, the chemotherapeutic agent is taxol. Nit2 gene or Nit2 protein also may be used in conjunction with radiotherapy. The type of ionizing radiation constituting the radiotherapy may be selected from the group comprising x-rays, gamma-rays and microwaves. In certain embodiments, the ionizing radiation may be delivered by external beam irradiation or by administration of a radionuclide. The Nit2 gene or Nit2 protein also may be used with other gene-therapy regimes. In particular embodiments the Nit2 gene is introduced into a tumor. The tumor may be in an animal, in particular, a human. The Nit2 gene or Nit2 protein may be introduced by injection.

According to the invention, the Nit2 gene or Nit2 protein of the method of the invention can be used in the inhibition of the tumor cell growth. Furthermore, the Nit2 gene can be used as gene therapy agent. Preferably, the following cancers can be treated through the method of the invention: melanoma, prostate, ovary, mammary, pancreatic, lungs, colon, smooth muscle, breast tumors.

The invention is also directed to a pharmaceutical composition for use in inhibiting tumor cell growth, which comprises a Nit2 gene and a pharmaceutically acceptable carrier.

The invention is also directed to a pharmaceutical composition for use in inhibiting tumor cell growth, which comprises a Nit2 protein and a pharmaceutically acceptable carrier.

According to the invention, the Nit2 gene is any DNA sequence or the fragment thereof that has the function equivalent to the natural Nit2 gene. For example, the DNA sequence includes: (a) the DNA analog sequence derived from coding regions of the natural Nit2 gene; or (b) the DNA analog sequence capable of hybridization of DNA sequences of (a) under moderately stringent conditions; or (c) the DNA sequences which are degenerative as a result of the genetic code to the DNA analog sequences defined in (a) or (b). Preferably, the Nit2 gene of the invention is a human Nit2 gene. More preferably, the Nit2 gene has the sequence as shown in SEQ ID NO: 1.

According, to the invention, the Nit2 proteins are those encoded by the Nit2 gene as described therein. Preferable, the Nit2 protein is that encoded by the sequence of SEQ ID NO: 1 or the degenerate sequence thereof.

According, to the invention, the Nit2 gene or Nit2 protein used in the pharmaceutical composition of the invention is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners.

In some embodiments of the present invention, the pharmaceutical composition comprising Nit2 gene or Nit2 protein can be used in combination with other anti-cancer therapies. These other therapies may be known at the time of this application, or may become apparent after the date of this application. Nit2 gene or Nit2 protein may be used in combination with other therapeutic polynucleotides, polypeptides or chemotherapeutic agents. For example, Nit gene or Nit2 protein may be used in conjunction with other known polypeptides, such as TNF-alpha, P53 or RB. Nit2 gene or Nit2 protein may be used in conjunction with any suitable chemotherapeutic agent. In one representative embodiment, the chemotherapeutic agent is taxol. Nit2 gene or Nit2 protein also may be used in conjunction with radiotherapy. The type of ionizing radiation constituting the radiotherapy may be selected from the group comprising x-rays, gamma-rays and microwaves. In certain embodiments, the ionizing radiation may be delivered by external beam irradiation or by administration of a radionuclide. The Nit2 gene or Nit2 protein also may be used with other gene-therapy regimes. In particular embodiments the Nit2 gene is introduced into a tumor. The tumor may be in an animal, in particular, a human. The Nit2 gene or Nit2 protein may be introduced by injection.

According to the invention, the pharmaceutical composition can be used in the inhibition of the following tumor cell: melanoma cell, prostate cell, ovary cell, mammary cell, pancreatic cell, lung cell, colon cell, smooth muscle cell, as well as the cell from glioblastoma, bone marrow stem cells, hematopoietic cells, osteoblasts, epithelial cells, fibroblasts.

The following Examples are offered by way of illustration and not by way of limitation.

### EXAMPLES

### Example 1

### Construction of the Plasmid pEGFP-C1-NIT2

Total RNA was derived from YM1 cells. The culture medium of the YM1 cell culture was removed and 1 ml Trizole reagent was added to the cells. After complete mixing, the cells were scraped into a 1.5 ml eppendorf and incubated at room temperature for 5 minutes. After the addition of 0.2 ml chloroform, the mixture was vortexed for 15 minutes, followed by incubation at room temperature for 2 minutes. The mixture was then centrifuged at 12000×g, 4° C. for 15 minutes, and the supernatant was removed to a new eppendorf. An equal volume of isopropanol was added. After complete mixing, the mixture was incubated at room temperature for 10 minutes, followed by centrifugation at 12000×g, 4° C. for 5 minutes. The resulted white pellet (precipitated RNA) was washed with 1 ml 75% ethanol, followed by centrifugation at 12000×g, 4° C. for 5 minutes. The supermatant was discarded, and the pellet was placed on the ice until dry. The dried pellet was dissolved in DEPC water and stored at −70° C. The quality of the RNA was confined by 0.8% 1×MOPS agarose gel electrophoresis analysis to observe the intensities of 18 s and 28 s rRNA.

The reverse transcription-polymerase chain reaction (RT-PCR) was carried out with 1-5 μg total RNA derived above, using Super Script Kit (Gibco BRL, California, U.S.A). NIT2 gene-specific primers were added to 2 μg of the resulted first strand of cDNA, which is used as the template, to carry out the following PCR reaction. The DNA was first denatured at 90° C. for 1 minute, followed by 30 cycles of: 90° C.×20 seconds of denaturation, about 60° C.×20 seconds of annealing, and 70° C.×1 minute of extension. The reaction was ended by 70° C.×3 minutes of extension. The PCR product was analyzed by 2.0% agarose gel electrophoresis.

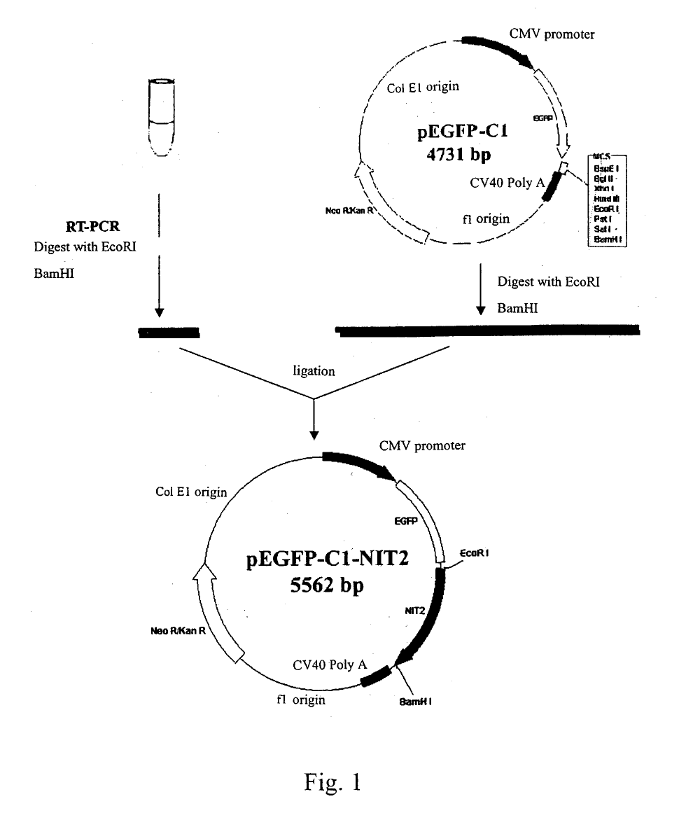

The PCR-amplified DNA was digested with restriction enzymes EcoRI and RamHI, purified with Gel Extraction Kit (Viogene), and then ligated into the plasmimd pEGFP-C1 (4731 bp) to obtain pEGFP-C1-NIT2 (5562 bp) (see FIG. 1). The plasrmid pEGFP-C1 comprises a sequence encoding the protein EGFP (green fluorescence protein), which emits green fluorescence.

### Example 2

### Expression of the EGFP-NIT2 Fusion Protein in OVCAR-3 Cancer Cells

The plasmid pEGFP-C1-NIT2 obtained in Example 1 was subsequently transformed into the host cells JM109 E. coli by adding the plasmid (10 μl of the ligated product) to 200 μl of competent cells. The cells were then placed on ice for 30 minutes, heat shocked in 42° C. water bath for 90 seconds, and rapidly placed back on ice for 2 minutes. The transformed cells were added with 800 μl of LB medium and cultured with shaking at 37° C. for 1 hour. The cell culture (200 μl) was applied to Amp⁺ LB medium plate and cultured at 37° C. for 16 to 18 hours until colonies appear. Mini-preparation of pEGFP-C1-NIT2 was carried out according to common molecular biology protocols.

Lipofection of OVCAR-3 cells (human ovary cancer cell-line) was carried out when the cells have grown to cover 80% of the area of the culture dish. Six μl of Lipofectamine 2000 and 1 μl of the plasmid prepared above were respectively diluted with 375 μl of serum free medium, and incubated at room temperature for 5 minutes. The two are then put together and mixed at room temperature for 30 minutes. The culture medium of OVCAR-3 cells was replaced with serum free medium, and the lipofectamine-plasmid mixture was added therein. After 24 hours, the serum free medium was replaced and relevant analyses were performed.

The Fhit gene and the protein thereof were used as control. The plasmid pEGFP-C1-FHIT was constructed using the procedure for constructing the plasmid pEGFP-C1-NIT2 of Example 1. The resulting plasmid pEGFP-C1-FHIT was also transfected into OVCAR-3 cells as control in the following Examples 3 and 4.

### Example 3

### Expression of Nit2 in Transfected Cancer Cells

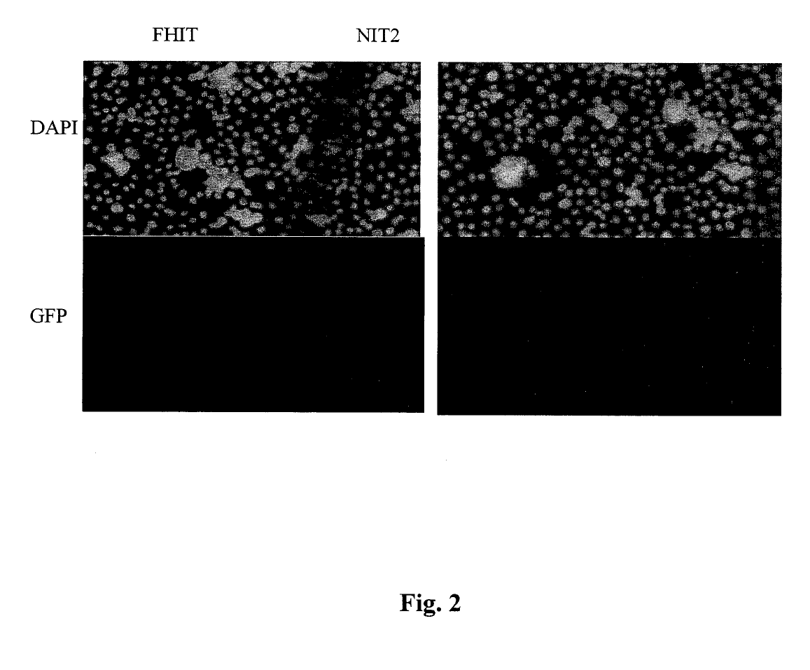

The transfected OVCAR-3 cells prepared in Example 2 were treated with DAPI solution (50 μg DAPI in 1 ml Tris-saline) and then collected by centrifuged under 700 g at 4° C. The resulting cells were washed by PBS buffer and then adjusted to the concentration of 1.0×10⁶cells/ml. The cells were fixed in slide using cytospin for two minutes and then solution of menthol and acetone (1:1) for five minutes. The slide was washed by Tris-saline buffer for five minutes. 40 μl of DAPI solution was added to the cells on the slide, which were then incubated without light at 37° C. for one hour. The resulting slide was washed by −20° C., 70% alcohol and 100% alcohol. After drying, 35 μl of mounting solution (10 mg of para-phenylenediamine, 1 ml, 1.5M of Tris-HC1 (pH8.8) and 9 ml of glycerol) was added to the slide. The cells on the slide were detected and photographed by fluorescence microscopy. As shown in FIG. 2, DAPI staining demonstrated the localization of nucleus of OVCAR-3 cancer cells. The green fluorescence indicated the expression of pEGFP-C1-Nit2 and pEGFP-C1-Fhit in cytosol of OVCAR-3 cells. Expression of FHIP (pEGFP-C1-Fhit) was employed as positive control.

### Example 4

### Determination of Growth Curve of the Cells

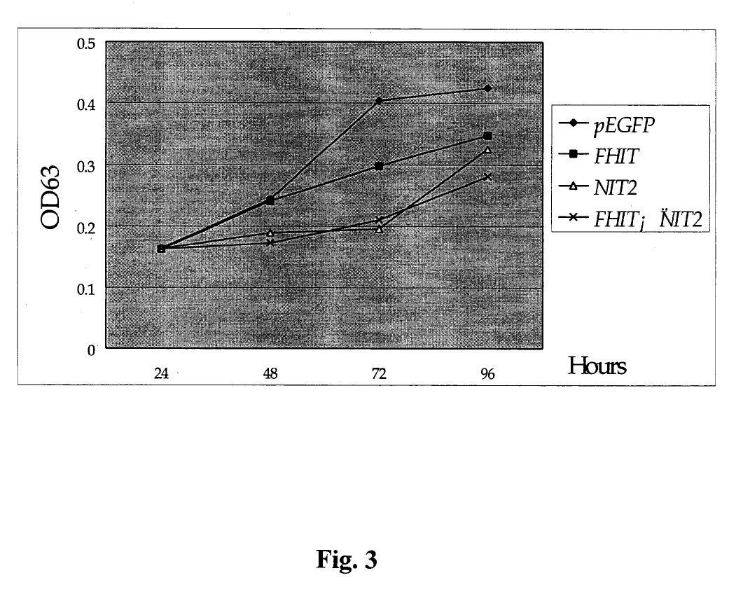

The transfected cells of Example 2 were inoculated in 96-well plate in a density of 0.5×10³³ cells/well. After the cells were incubated for 48 hours, the culture medium was changed with serum free medium and then 25 μl of dye (MTT (3-[4,5 dimethylthiazol-2-yl] 2,5-diphenyltetrazolium bromide) solution) was added to the cells for 4 to 8 hours. Then, 200 μl of lysis buffer (200 μl of DMSO and 25 μl of Sorensen's glycine buffer) was added to the cells to dissolve the blue-purple crystals. The absorbance of the resulting cells was determined at 570 nm by ELISA reader. As shown in FIG. 3, pEGFP transfected in OVCAR-3 is a blank study. The growth curve of pEGFP-FHIT transfected cells is the positive control. The growth rate of pEGFP-Nit2 transfected cell was 50% of that of pEGFP transfected OVCAR-3 cells after 72 hours of transfection.

### Example 5

### Determination of Cell Apoptosis by Flow Cytometry

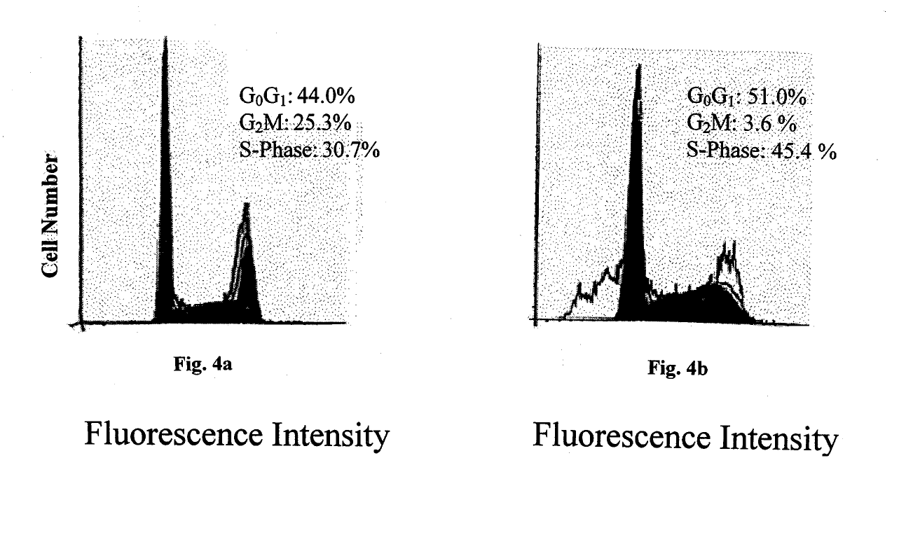

Apoptosis and cell cycle analysis of transfected cells from Example 2 was determined by Flow Cytometry, FACS according to the method as described in Telford et al., Cell Prolif. 24, 447-459. The percentage of G0/G1, G2 and S phase of transfected OVCAR-3 was analyzed by computer software. In FIG. 4(b), the increased G0/G 1 percentage (51%) and sub-G0 peak compared with that in FIG. 4(a) indicated the presence of apoptotic cells.

### Example 6

### Inhibition of Humor Growth of Nude Mice by Adenovirus Vector-Mediated Human Nit2 Gene Expression

Homologus Recombination of Adenoviral Plasmid Vector and a Shuttle Vector Carrying Nit 2 cDNA

Human Nit2 cDNA was cloned into a shuttle vector, pShuttle-CMV with Stratagene (CA. USA) AdEasy adenoviral vector system. The forward primer used is GGAAGATCTATGACCTCTTTCCGC. The reversed primer used is CAGCTCGAGTTAGGGCTTTTTCATC. The resultant plasmid pShuttle-CMV-NIT2 was linearized by Pme1 and cotransformed into E. coli strain BJ5183 with the adenoviral plasmid pAdEasy-1. Recombinant adenoviral plasmids were selected on kanamycin. The viral vector Ad-Nit2 was obtained. The shuttle vector pShuttle-CMV-GFP was also prepared to validate the AdEasy-1 system.

Preparation of Primary Adenovirus Stock with Recombinant Adenovirus Plasmid

The recombinant adenoviral plasmids were then digested with Pac1 and transfected into HEK 293 cells in which recombinant adenoviral plasmids were packaged.

In vivo Mouse Experiment

Five mice were raised for each treatment group. To study the effect of Nit2 on tumor growth, SKOV-3 ovarian cancer cells and Hep G2 cells were used to establish s. c. tumors in nude mice, respectively. 1×10⁷ SKOV-3 cells or 1×10⁶ HepG2 cells in 100 ml of PBS were injected into the right flank of female nude mice, 7-8 weeks of age. When the tumors reached 7-9 mm in diameter, 100 ml of PBS containing Ad-Nit2 or control vectors was injected into the center of the tumor for four times at days 1, 4, 8 and 11, at a total dose 3×10¹⁰pfu/tumor. A viral vector expressing GFP, Ad-CMV-GFP, was used to monitor transduction efficiency. Ad-E1-GAL4(Ad-EV), an empty E1 vector, was used as a negative control. PBS alone was used as a mock treatment control. Tumor dimensions were monitored in length and width by using calipers. The tumor volume was calculated using V(mm³)=a×b²/2, where “a” is the largest dimension and “b” is the perpendicular diameter. The in vivo results were expressed as “mean±SD” in five mice for each treatment group. Student's two-sided t test was used to compare the value.

The growth of tumors was recorded from the first injection until 20 days after last injection for SKOV-3 or HepG2 cells s.c. tumor model. All of the tumors in the mice treated with Ad-Nit2 showed significantly suppressed growth of tumors bearing SKOV-3 cells, or HepG2 cells tumor models, compared with tumors in Ad-EV treated mice or PBS treated control. The result showed that the tumors injected with Ad-Nit2 were only about one tenth of the size of the tumors injected with Ad-EV vector or tumors injected with PBS alone.

What is claimed is:

1. A pharmaceutical composition for use in inhibiting tumor cell growth in a mammal, which comprise a Nit2 gene in an amount effective to inhibit tumor cell growth and pharmaceutically acceptable carrier.

2. The pharmaceutical composition of claim 1, wherein the mammal is human.

3. The pharmaceutical composition of claim 1, wherein the Nit2 gene is a human Nit2 gene.

4. The pharmaceutical composition of claim 1, wherein the Nit2 gene has the sequence as shown in SEQ ID NO: 1.

5. The pharmaceutical composition of claim 1, wherein the tumor cell is selected from the group consisting of: melanoma cell, prostate cell, ovary cell, mammary cell, pancreatic cell, lung cell, colon cell, smooth muscle cell, as well as the cell from glioblastoma, bone marrow stem cells, hematopoietic cells, osteoblasts, epithelial cells, fibroblasts.

6. The pharmaceutical composition of claim 1, which can be used in the treatment of melanoma, prostate, ovary, pancreatic, lungs, colon, smooth muscle and breast tumors.

7. The pharmaceutical composition of claim 1, which can further comprises other therapeutic anti-cancer polynucleotides, polypeptides or chemotherapeutic agents.

8. A pharmaceutical composition for use in inhibiting tumor cell growth in a mammal, which comprise a Nit2 protein in an amount effective to inhibit tumor cell growth and pharmaceutically acceptable carrier.

9. The pharmaceutical composition of claim 8, wherein the mammal is human.

10. The pharmaceutical composition of claim 8, wherein the Nit2 protein is encoded by the sequence as shown in SEQ ID NO: 1 or the degenerate sequence thereof.

11. The pharmaceutical composition of claim 8, wherein the tumor cell is selected from the group consisting of: melanoma cell, prostate cell, ovary cell, mammary cell, pancreatic cell, lung cell, colon cell, smooth muscle cell, as well as the cell from glioblastoma, bone marrow stem cells, hematopoietic cells, osteoblasts, epithelial cells, fibroblasts.

12. The pharmaceutical composition of claim 8, which can be used in the treatment of melanoma, prostate, ovary, pancreatic, lungs, colon, smooth muscle and breast tumors.

13. The pharmaceutical composition of claim 8, which can further comprises other therapeutic anti-cancer polynucleotides, polypeptides or chemotherapeutic agents.
